SEQUENCE LISTING (2 sequences)

SEQ ID NO: 1 - protein, 118 aa, Homo sapiens
  4EBP1
MSGGSSCSQTPSRAIPATRRVVLGDGVQLPPGDYSTTPGGTLFSTTPGGTRIIYDRKFLMECRNSPVTKTPPRDLPTIPGVTSPSSDEPPMEASQSHLRNSPEDKRAGGEESQFEMDI

SEQ ID NO: 2 - protein, 217 aa, Homo sapiens [sequence omitted]

(19) United States
(12) Patent Application Publication
(10) Pub. No.: US 2024/0052004 A1
(43) Pub. Date: Feb. 15, 2024

(54) TARGETING CAP-DEPENDENT TRANSLATION TO REDUCE SEIZURES IN MTOR DISORDERS

(71) Applicant: Yale University, New Haven, CT (US)

(72) Inventors: Angelique BORDEY, Guilford, CT (US); Lena NGUYEN, New Haven, CT (US); Lawrence HSIEH, Hartford, CT (US); Tiffany LIN, New Haven, CT (US)

(21) Appl. No.: 17/766,455

(22) PCT Filed: Oct. 2, 2020
(86) PCT No.: PCT/US2020/054007

§ 371 Date: Apr. 4, 2022

Related U.S. Application Data
(60) Provisional application No. 62/910,749, filed on Oct. 4, 2019.

(51) Int. Cl. C07K 14/47; C12N 9/22; A61P 25/08; C12N 15/86

(52) U.S. Cl. CPC C07K 14/4702; C12N 9/22; A61P 25/08; C12N 15/86; C12N 2310/20

(57) ABSTRACT

In various aspects and embodiments the invention provides a method of preventing seizures in a subject in need thereof, the method comprising contacting a target cell of the subject with a 4EBP-activating agent or a EIF4E-depleting agent.

### CROSS-REFERENCE TO RELATED APPLICATIONS

This application claims priority under 35 U.S.C. § 119(e) to U.S. Provisional Patent Application No. 62/910,749, filed Oct. 4, 2019, all of which is hereby incorporated by reference in its entirety herein.

### STATEMENT REGARDING FEDERALLY SPONSORED RESEARCH OR DEVELOPMENT

This invention was made with government support under NS086329 and HD095567 awarded by National Institutes of Health. The government has certain rights in the invention.

### BACKGROUND OF THE INVENTION

Diseases involving mTOR activation, such as focal cortical dysplasia type II (FCDII) and tuberous sclerosis complex (TSC), are neurodevelopmental disorders caused by mutations in the PI3K-mTOR pathway and GATOR complex genes leading to mTOR hyperactivity, focal malformations of the developing cortex, and seizures in 80-90% of the patients. Nearly two-thirds of these patients are refractory to treatment with anti-epileptic drugs and experience life-long seizures, leading to a spectrum of neurocognitive and psychological disabilities. The current treatments are surgical resection of the focal cortical malformations and administration of anti-epileptic drugs, including in TSC, everolimus, a rapamycin analog, which inhibits mTOR activity. These treatments are either invasive or are associated with serious adverse events, and neither is fully effective. There is thus a critical need to improve epilepsy treatment in patients suffering from mTOR hyperactivation, such as TSC and FCDII patients. This disclosure addresses that need.

### BRIEF SUMMARY OF THE INVENTION

The present disclosure relates in part to a method of preventing seizures in a subject in need thereof, the method comprising contacting a target cell of the subject with an effective amount of a 4EBP-activating agent or a EIF4E-depleting agent. In certain embodiments, the seizures are associated with mTOR hyperactivity.

In certain embodiments, the 4EBP-activating agent comprises a viral vector comprising a polynucleotide encoding a constitutively active form of 4EBP (4EBP^{CA}) In certain embodiments, the 4EBP-activating agent is a viral vector comprising a CRISPR system configured to mutate 4EBP into a constitutively active form.

In certain embodiments, the EIF4E-depleting agent is dominant negative EIF4E. In certain embodiments, the EIF4E-depleting agent comprises a viral vector comprising a EIF4E-inhibitory polynucleotide. In certain embodiments, the EIF4E-inhibitory polynucleotide is an EIF4E or EIF4G antisense oligonucleotide, an EIF4E or EIF4G small hairpin RNA (shRNA), an EIF4E or EIF4G small-interfering RNA (siRNA) or a CRISPR system comprising a guide RNA targeting EIF4E or EIF4G. In certain embodiments, the EIF4E-depleting agent is a EIF4E inhibitor selected from the group consisting of: Bn7GMP, 4Ei-1, 4EGI-1 and 4E1RCat.

In certain embodiments, the 4EBP-activating agent or the EIF4E-depleting agent is directly injected into the brain of the subject. In certain embodiments, the 4EBP-activating agent or EIF4E-depleting agent is directly injected at a site of focal cortical malformations.

In certain embodiments, the viral vector further comprises a tissue specific promoter. In certain embodiments, the viral vector is an adenoviral vector or a lentiviral vector. In certain embodiments, the target cell is a neuron. In certain embodiments, the subject or neuron displays focal cortical malformations.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a a simplified diagram of a biochemical pathway that involve genes mutated in several neurodevelopmental disorders. Disorders with mutations in: TSC1 or TSC2—Tuberous sclerosis complex; NF1—Neurofibromatosis 1; PI3K, AKT-Hemimegencephaly; mTOR, TSC1, TSC2—Focal Cortical Dysplasia; PTEN—Cowden syndrome; mTOR—Focal cortical dysplasia; DEPDC5 and Npr12/3-, GATOR genes associated mTOR disorders. Mutations in these genes lead to hyperactive mTORC1, and a spectrum of neurological deficits, including (but not limited to) cognitive dysfunction, autism-relevant behaviors and mild to severe psychiatric disabilities, and abnormalities of cortical development.

FIG. 2 depicts tuberous sclerosis complex (TSC) and focal cortical dysplasia (FCD), two common classes of malformations of cortical development (MCD). Subjects with TSC may demonstrate cognitive defects and autism, whereas subjects with FCD may demonstrate gross abnormalities, epilepsy, cognitive and psychiatric defects. In mTOR-related disorders, most if not all patients will contain somatic mutations that occur during development. These somatic mutations lead to malformation of cortical development. Malformation are focal like in TSC or FCD type 2. In addition, TSC individuals are often born with a germline mutation in addition to the somatic mutations occurring during embryonic life.

FIG. 3A is a diagram depicting the generation of behavioral symptoms in mice. FIG. 3B depicts the process of in utero electroporation (IUE) of Rheb in the medial prefrontal cortex (mPFC) of mice.

FIGS. 4A-4E demonstrate that Rheb^{CA}-induced focal malformations in the mPFC induce spontaneous seizures. FIG. 4A: Images of coronal sections containing neurons electroporated with control plasmid in the mPFC with an expanded electroencephalography (EEG) and electromyography (EMG) trace. FIG. 4B: Images of coronal sections containing neurons electroporated with Rheb^{CA} in the mPFC with an expanded EEG and EMG trace. FIG. 4C: Experimental setup for video, EEG, and EMG monitoring of mice with either a control plasmid or Rheb^{CA} in the mPFC. FIG. 4D: Bar graphs showing the number of seizures/day from the control and Rheb^{CA}-expressing cohorts (*P<0.05). FIG. 4E: Bar graph showing the seizure duration in animals with seizures (n=8) from the mPFC Rheb^{CA}-expressing cohort (*P<0.05).

FIGS. 5A-5D depict TORC1-induced neuronal misplacement and dysmorphogenesis, wherein 70-80% of neurons are misplaced, dendritic trees are more complex, and spine density is decreased. FIG. 5A: Anterior cingulate cortex. FIG. 5B: Images of 2/3 GFP⁺ cells in coronal sections from P28 control and Rheb^{CA}-electroporated mice (scale bar: 50 μm). FIG. 5C: Sholl analysis and representative traces of dendrites (n=5-7 cells, three or four mice per condition, RM two-way ANOVA, Bonferroni post hoc). FIG. 5D: Spine density quantification and images of basal dendrites in control and Rheb^{CA} neurons (n=9-13 branches, three mice per condition, one-way ANOVA, Tukey post hoc, magnification: 100×, *P<0.05).

FIG. 6A depicts a diagram of the mTOR downstream outcome of normalizing cap-dependent translation using 4E-BP (increased soma size, neuronal misplacement, partially dendritic defects—not spines, and axonal defects) or reducing S6K levels (S6K1/2 shRNA prevented—axonal defects, but not misplacement). FIG. 6B: Summary of constructs used in study of cap-dependent translation. FIG. 6C: Cap-dependent translation as measured by a dual luciferase reporter in Neuro2a cells (n=3 replications per condition, one-way ANOVA, Tukey post hoc, RLU—relative light units, ***P<0.001, ****P<0.0001). FIG. 6D: Expression of 4EBP1^{CA} blocks cap-dependent translation but does not reduce pS6 increase induced by Rheb^{CA}−pS6 IF is still elevated in 4EBP1^{CA}+Rheb^{CA} electroporated cells relative to unelectroporated cells in the contralateral hemisphere (n=26-32 cells per hemisphere, three mice per condition, ****P<0.0001).

FIGS. 7A-7H demonstrate that normalizing protein translation prevents most mTOR-induced defects. FIG. 7A: Images of layer 2/3 GFP⁺ cells from BFP+Rheb^{CA} and 4EBP1^{CA}+Rheb^{CA} electroporated mice at P24 and soma area quantification (n=37-40 cells, three mice per condition t test, scale bars—40 μm, ****P<0.0001). FIG. 7B: Sholl analysis and representative traces of dendrites (n=5-7 cells, three or four mice per condition, RM two-way ANOVA, Bonferroni post hoc). Shaded areas indicate radii where P<0.05. Shaded region (a) indicates Rheb^{CA} vs. 4EBP1^{CA}+Rheb^{CA} comparison; shaded region (b), 4EBP1^{CA}+Rheb^{CA} vs. BFP; shaded region (c) Rheb^{CA} vs BFP. FIG. 7C: Images of GFP⁺ neurons in coronal sections from P7 mice in BFP+Rheb^{CA} and 4EBP1^{CA}+Rheb^{CA} conditions.

FIG. 7D: Percent of electroporated cells integrating in layer 2/3 is largely restored in the 4EBP1^{CA}+Rheb^{CA} condition (n=3 mice per condition, t test, ***P<0.001). FIG. 7E: Bar graph of the longest axon length in the corpus callosum (CC, n=6 axons with Rheb^{CA} and 8 axons with Rheb^{CA}+4EBP1^{CA}, n=3 mice, **P<0.01). FIG. 7F: Bar graph of the longest axon length in grey matter (n=6 axons with Rheb^{CA} and 6 axons with Rheb^{CA}+4EBP1CA, **P<0.01). FIG. 7G: Confocal images of tdTomato-fluorescent axonal projections from ACC neurons electroporated at E15 with Rheb^{CA}+GFP. FIG. 7H: Confocal images of tdTomato-fluorescent axonal projections from ACC neurons electroporated at E15 with Rheb^{CA}+4EBP1CA FIGS. 8A-8P: Decreasing cap-dependent translation via conditional 4EBP1^{CA} expression reduces mTOR-induced cellular abnormalities and seizures in mice. FIG. 8A: Schematic of experimental design. FIG. 8B: Immunofluorescence images of electroporated (tdTomato+) cells in the Rheb^{CA}+cGFP (top) and Rheb^{CA}+c4EBP1^{CA} (bottom) conditions.

FIG. 8C: Immunofluorescence images showing tdTomato+ cell placement in the cortex of Rheb^{CA}+cGFP (top) and Rheb^{CA}+c4EBP1^{CA} (bottom)-electroporated mice. FIG. 8D: Bar graph showing quantification of tdTomato+ cell soma size. n=11 Rheb^{CA}+cGFP and 20 Rheb^{CA}+c4EBP1^{CA} mice, ***P<0.0001 by Student's t-test (50 cells measured per animal). FIG. 8E: Bar graph showing quantification of tdTomato+ cell placement in the cortex. n=11 Rheb^{CA}+cGFP and 20 Rheb^{CA}+c4EBP1^{CA} mice, **P=0.0021 by Student's t-test. FIG. 8F: Representative EEG traces showing seizure activity in a Rheb^{CA}+cGFP-electroporated mouse. FIG. 8G: Pie chart showing the percentage of animals with or without seizures in each experimental group. FIG. 8H: Bar graph showing the average number of seizures per day during 7 days of consecutive EEG recording. n=11 Rheb^{CA}+cGFP and 18 Rheb^{CA}+c4EBP1^{CA} mice, *P=0.0260 by Mann-Whitney test. FIG. 8I: Scatterplot of seizures/day vs. cell soma size. n=29 XY pairs, P<0.0001 by Spearman's correlation. FIG. 8J: Heatmap showing the distribution of seizure frequency in the light and dark cycles during 7 days of consecutive EEG recording. White squares denotes cycles with no data. FIGS. 8K-8M: Data for the Rheb^{CA}+cGFP group. Note that only animals with seizures were included. FIG. 8K: Bar graph showing the percent of seizures in the light and dark cycles for each animal. FIG. 8L: Paired dot plots showing the total number of seizures that occur during the light or dark cycle for each animal. n=10 mice, *P=0.0313 by Wilcoxon matched-pairs signed rank test. FIG. 8M: Bar graphs showing the average number of seizures per 12 h-long light and dark cycles. n=10 mice, *P=0.0391 by Wilcoxon matched-pairs signed rank test. FIGS. 8N-8P: Data for the Rheb^{CA}+c4EBP1^{CA} group. Note that only animals with seizures were included. FIG. 8N: Bar graph showing the percent of seizures in the light and dark cycles for each animal. FIG. 8O: Paired dot plots showing the total number of seizures that occur during the light or dark cycle for each animal. n=10 mice, P=0.3613 by Wilcoxon matched-pairs signed rank test. FIG. 8P: Bar graphs showing the average number of seizures per 12 h-long light and dark cycles. n=10 mice, P=0.3340 by Wilcoxon matched-pairs signed rank test.

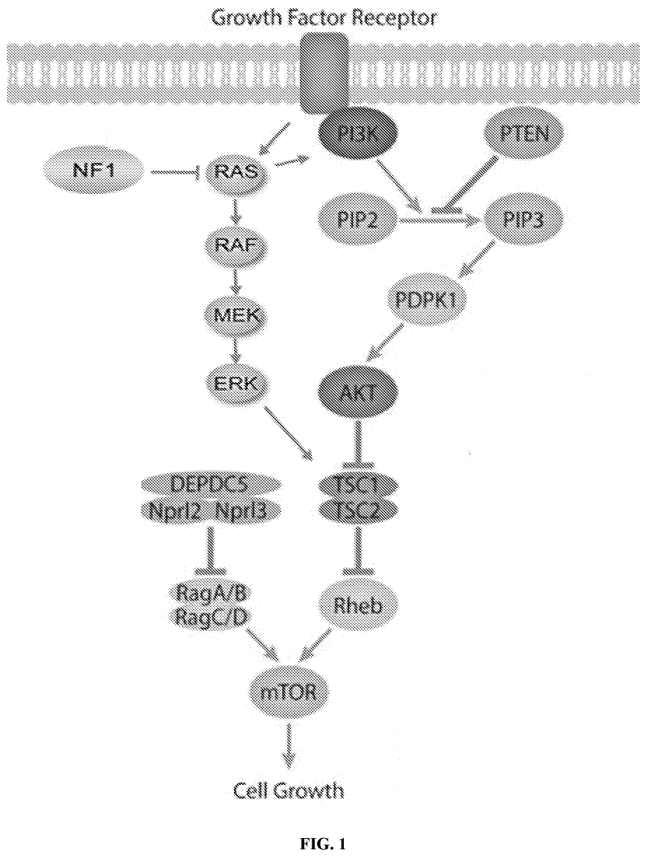

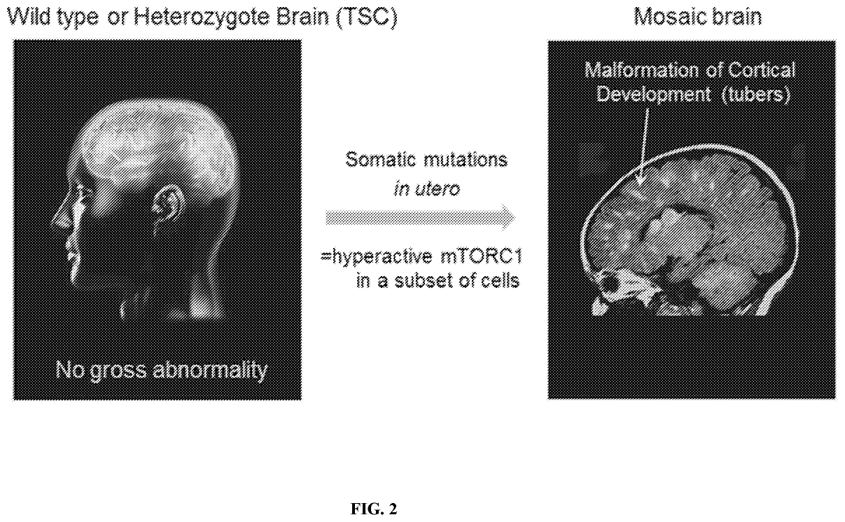

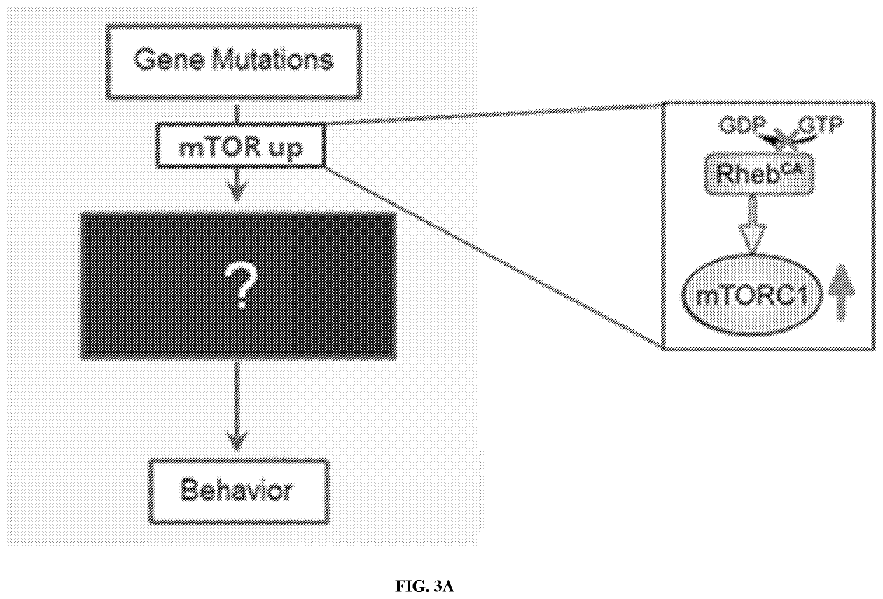

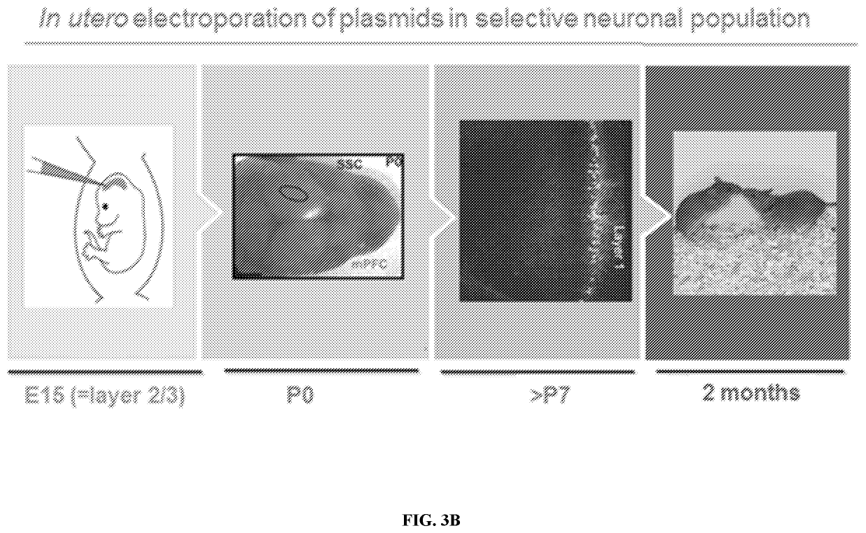

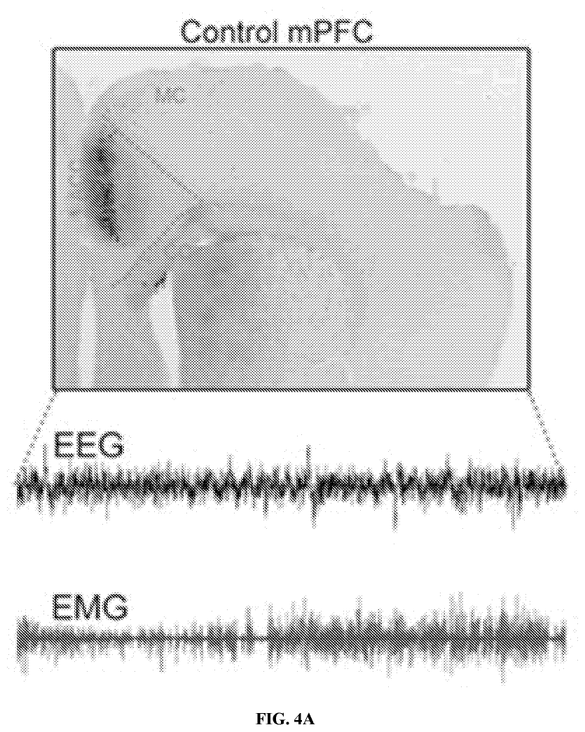

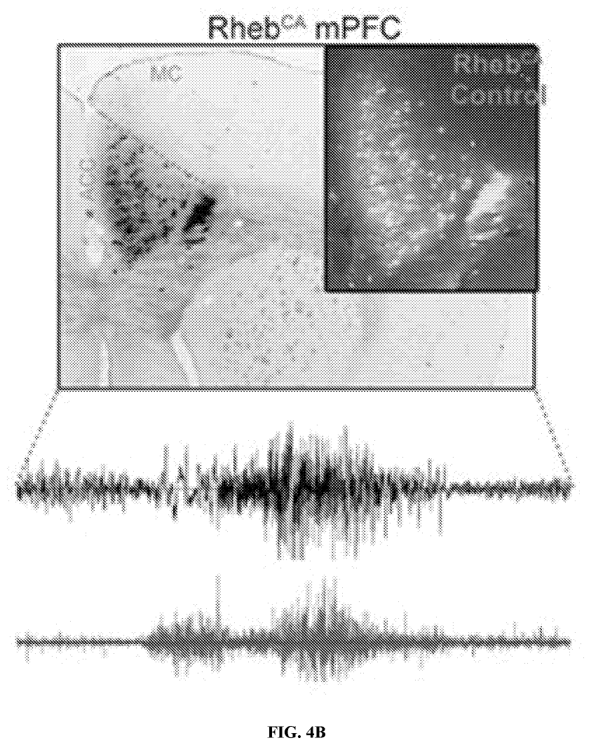

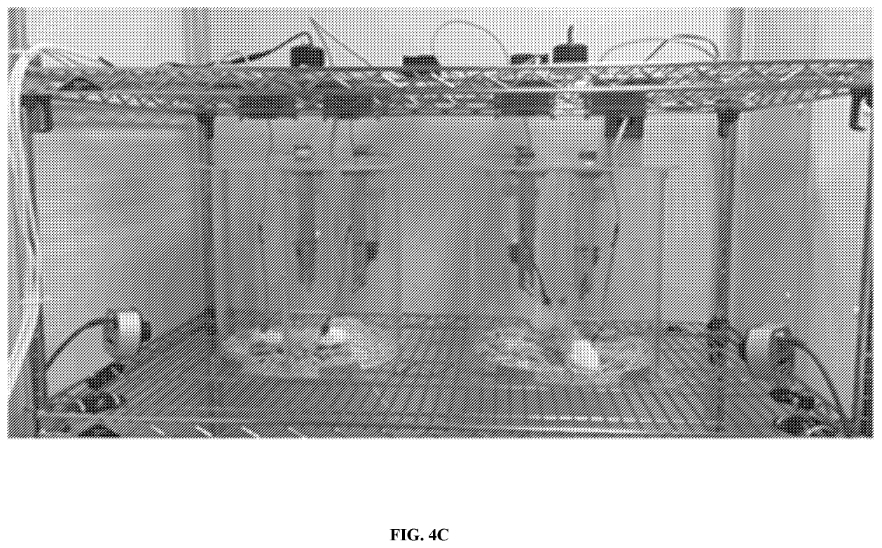

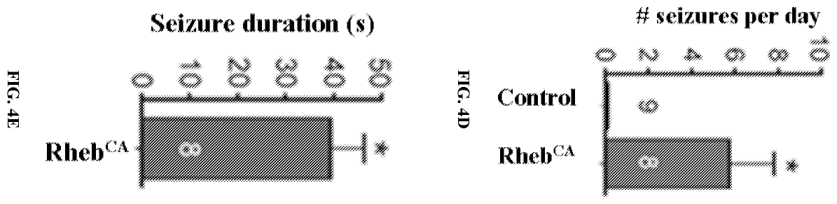

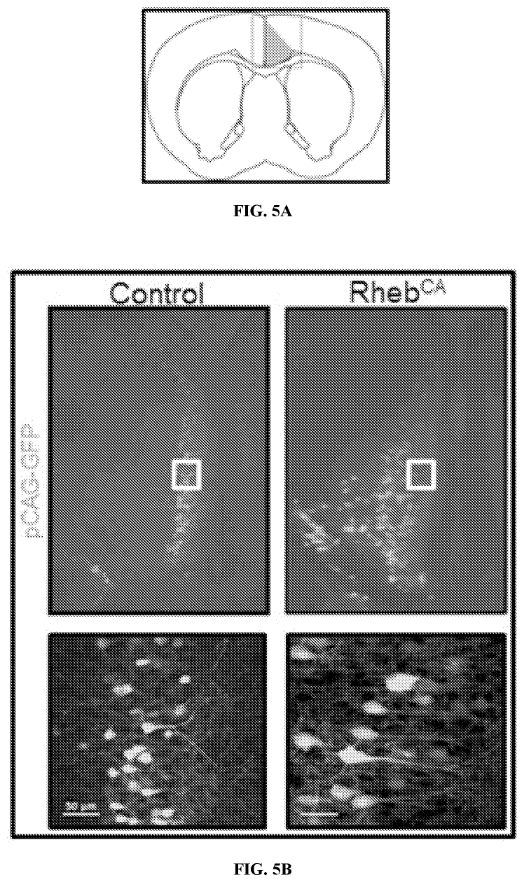

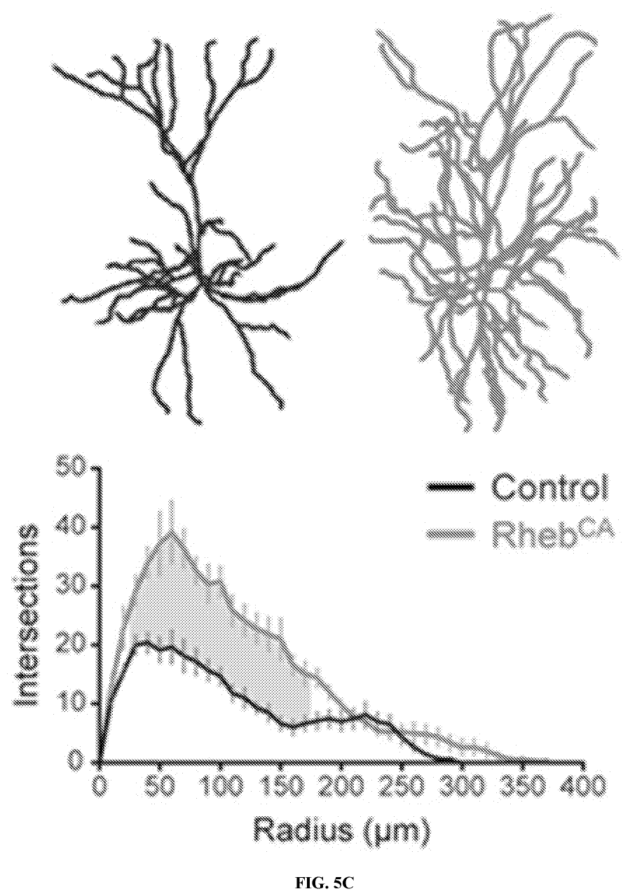

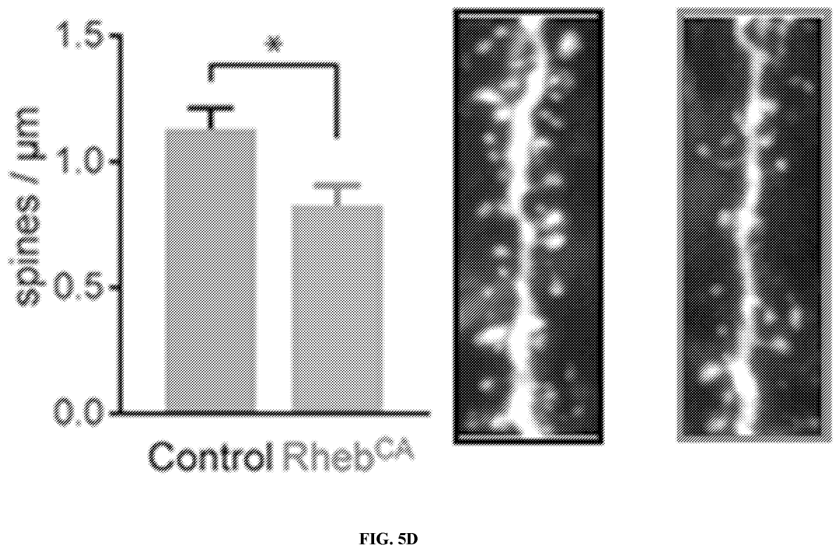

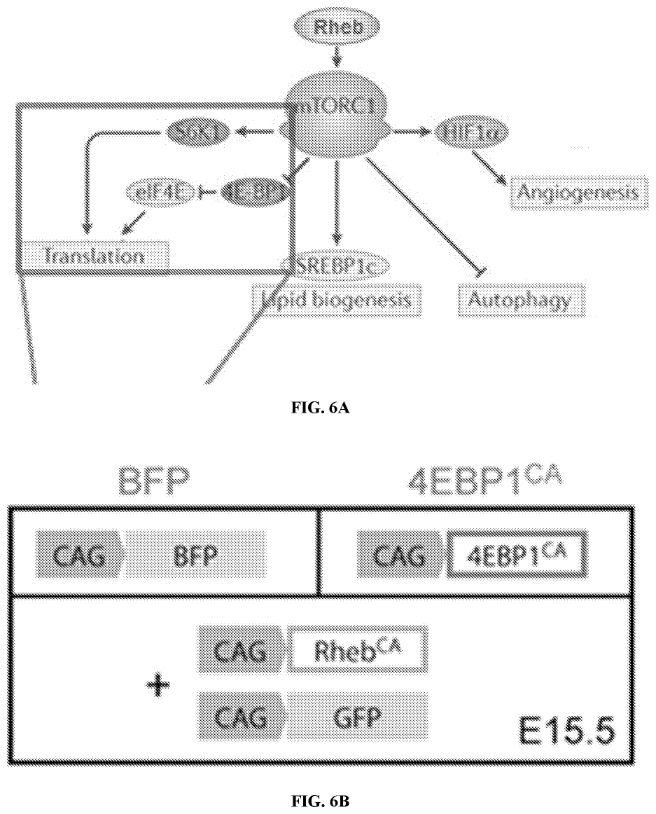

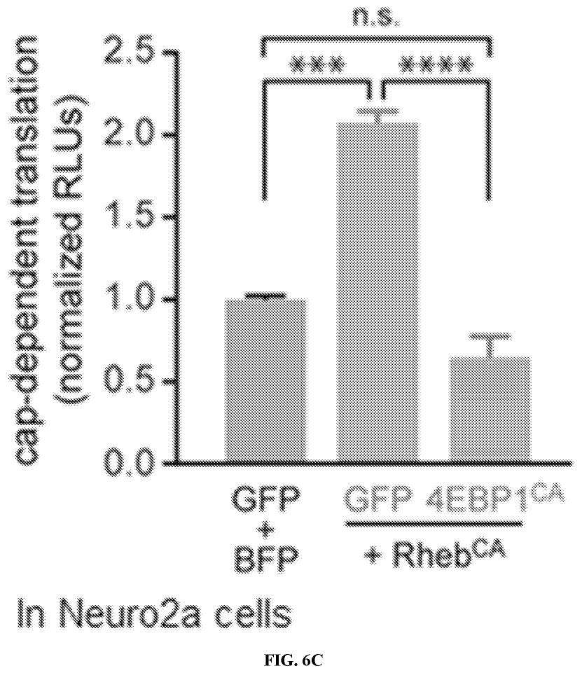

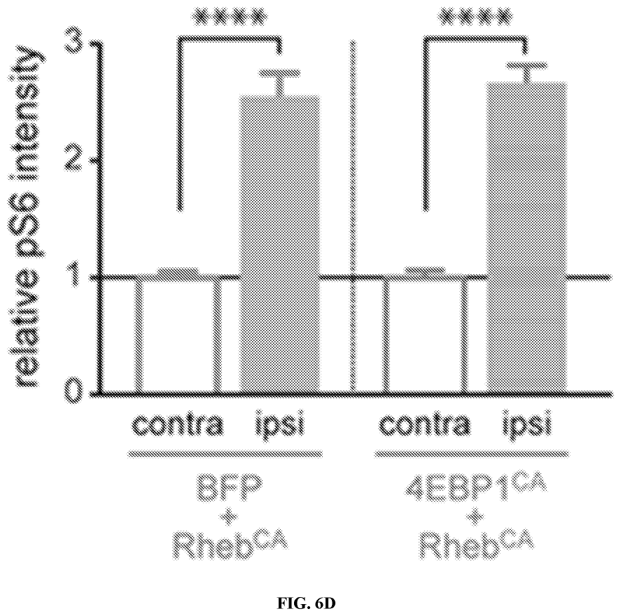

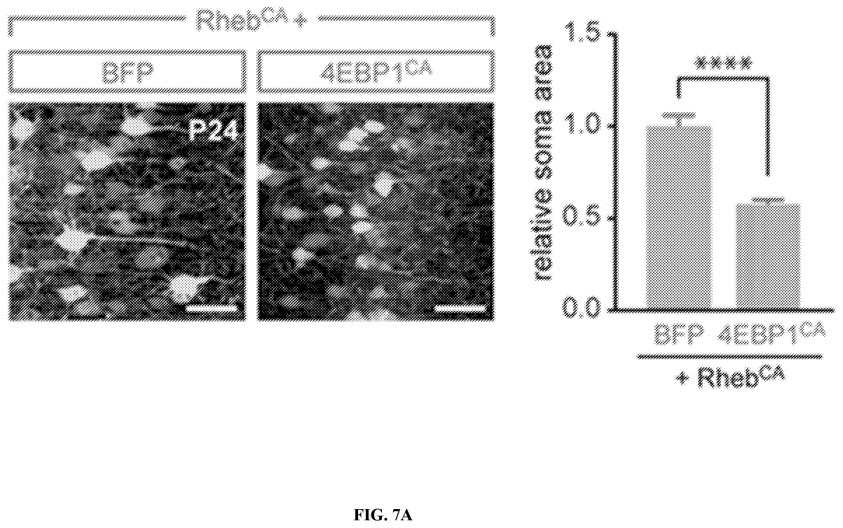

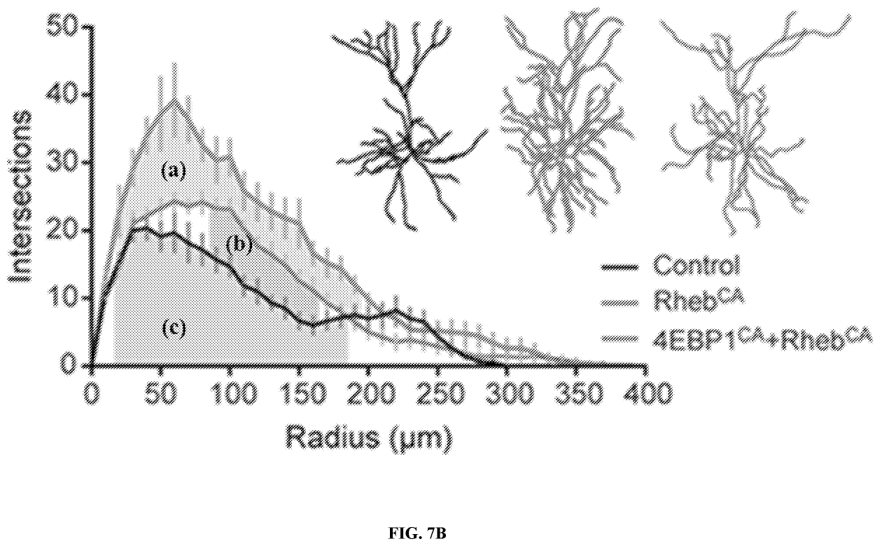

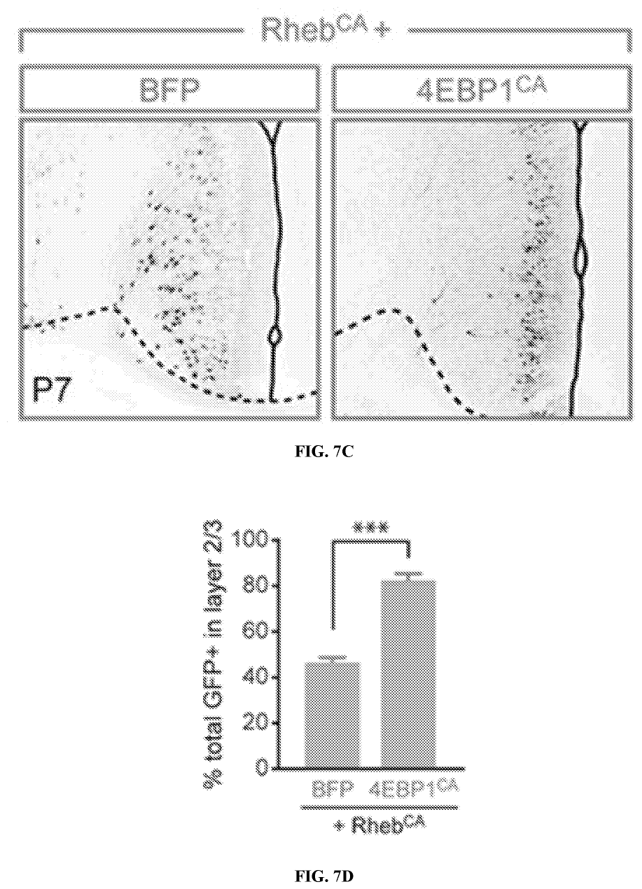

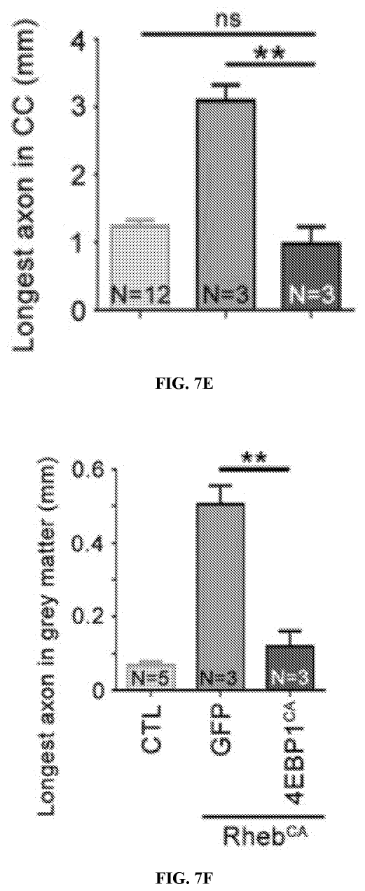

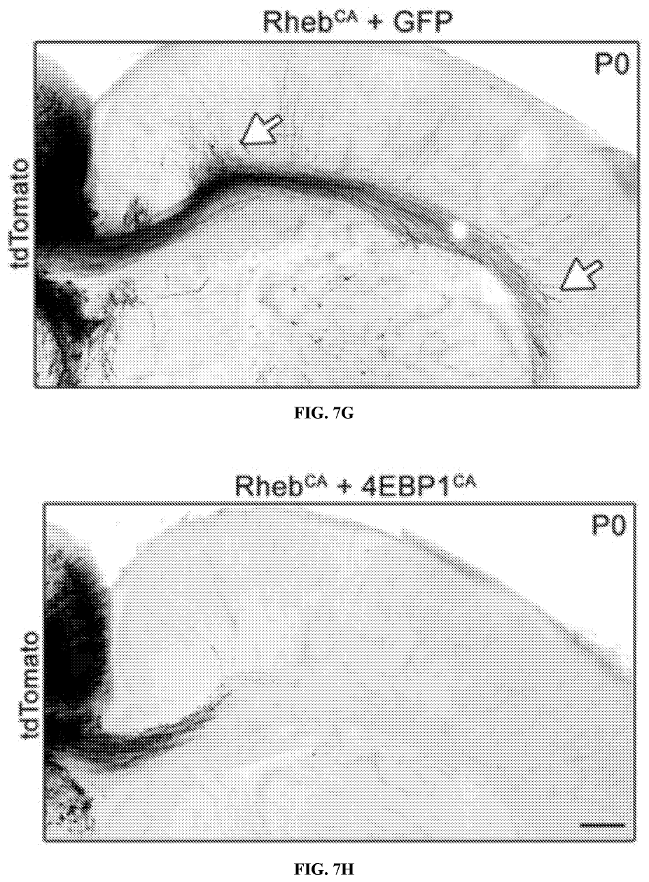

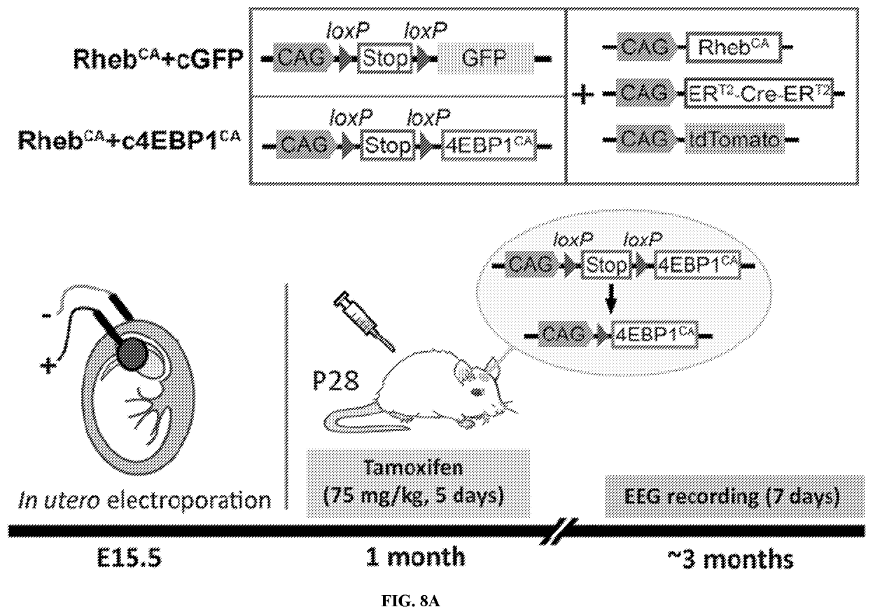

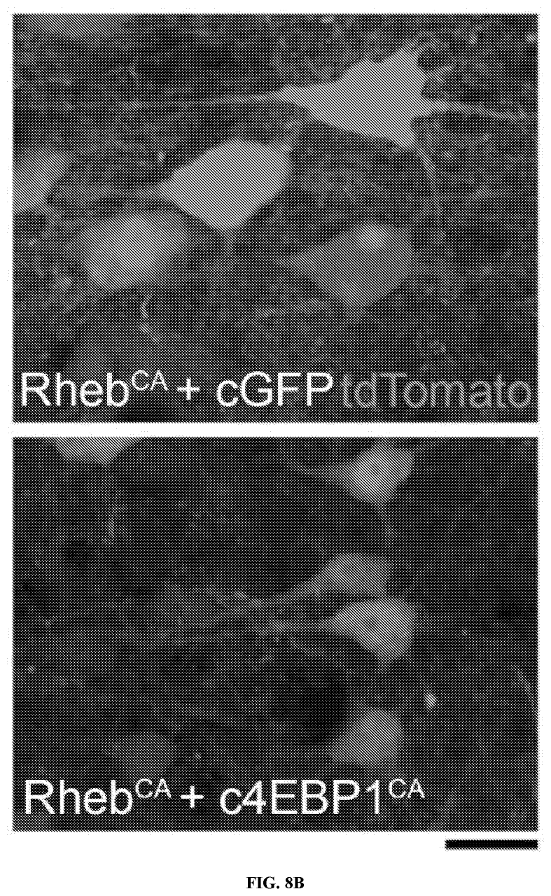

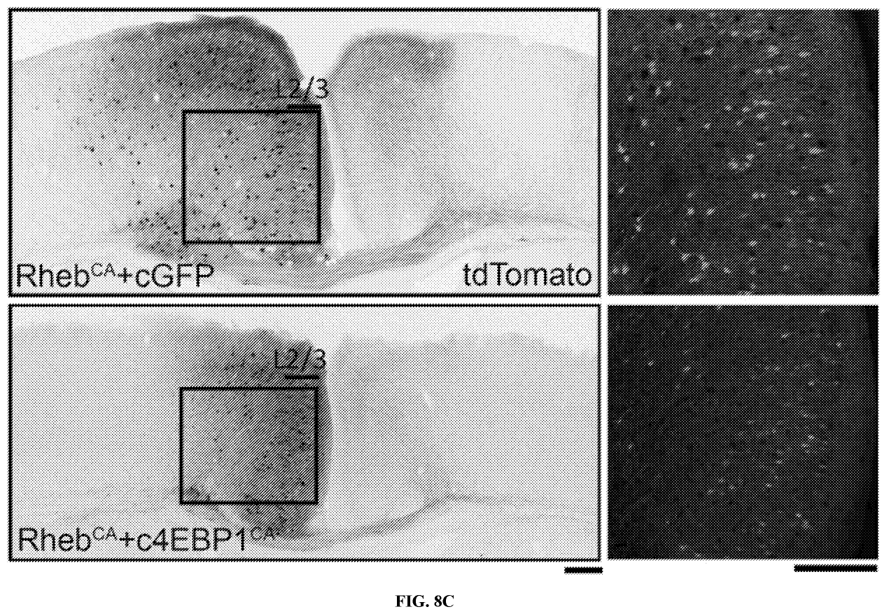

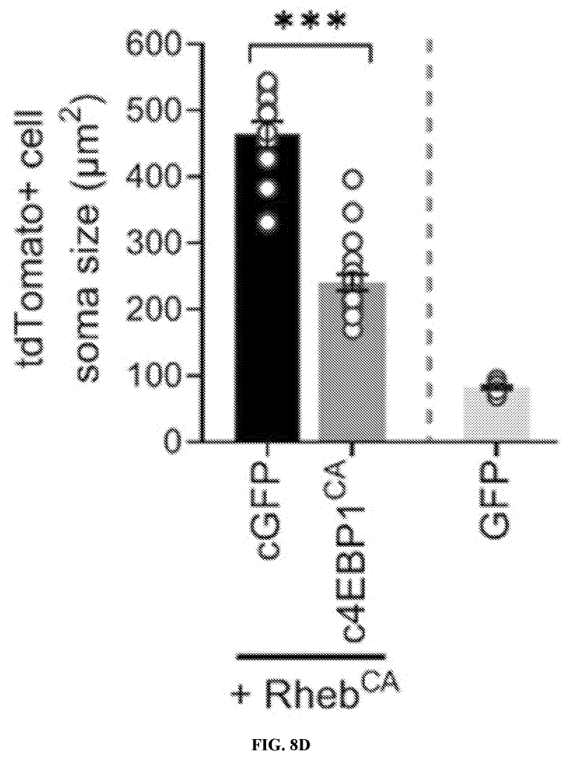

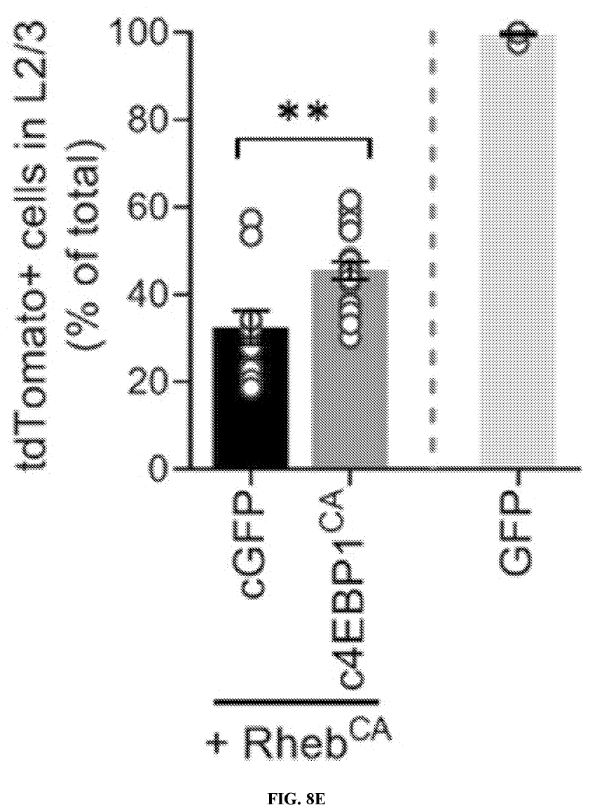

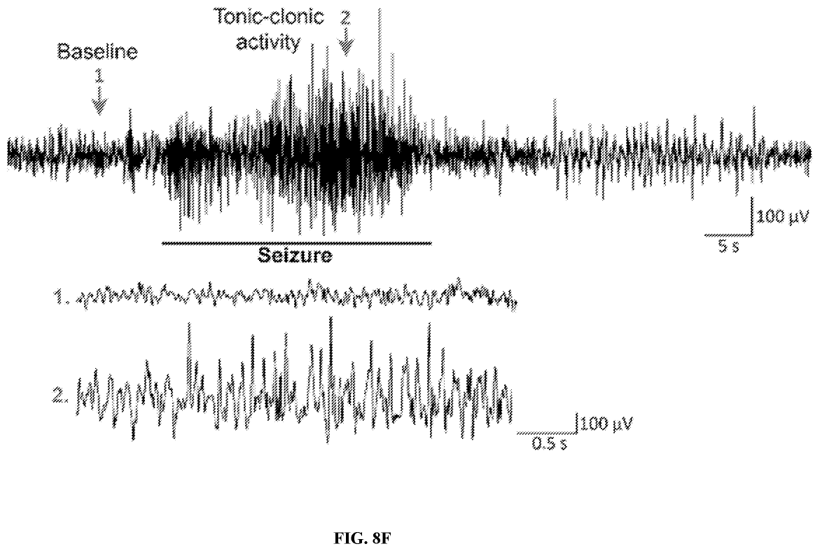

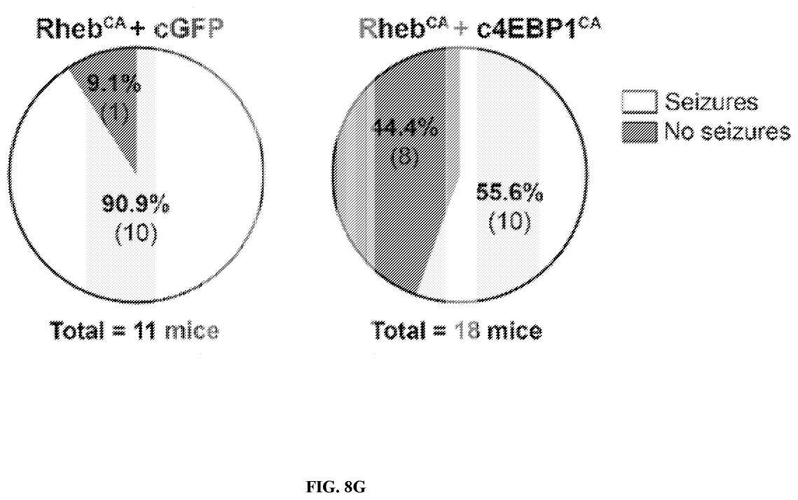

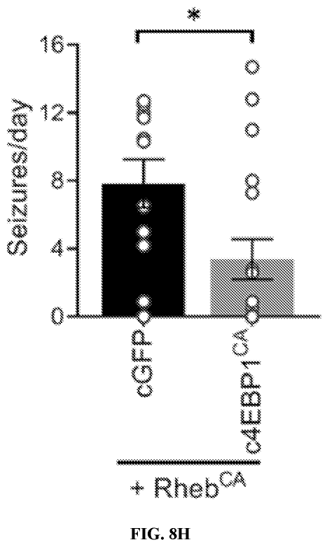

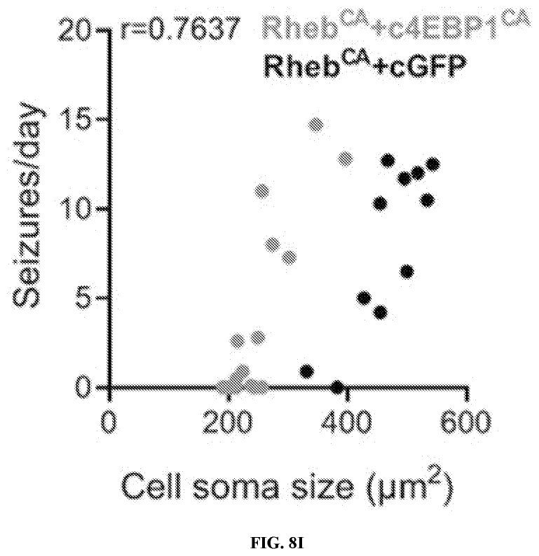

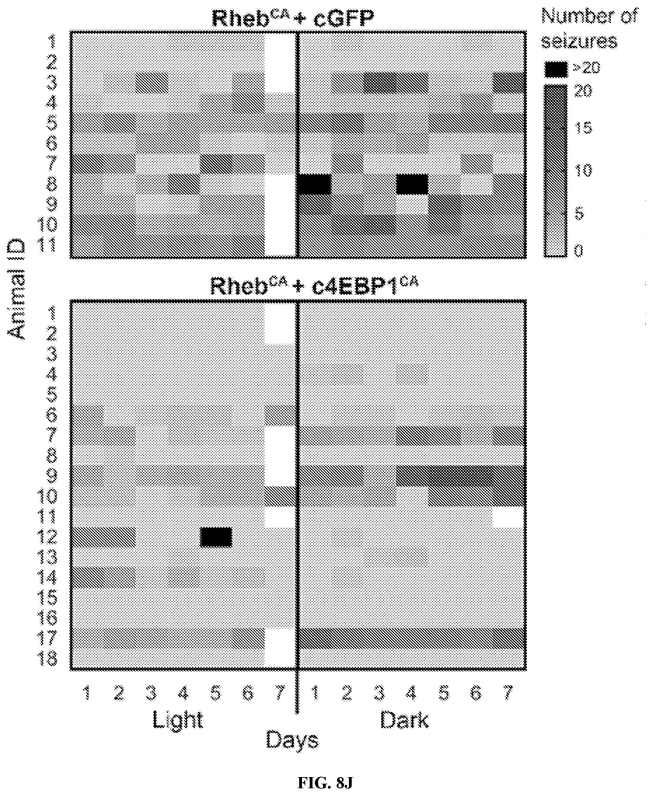

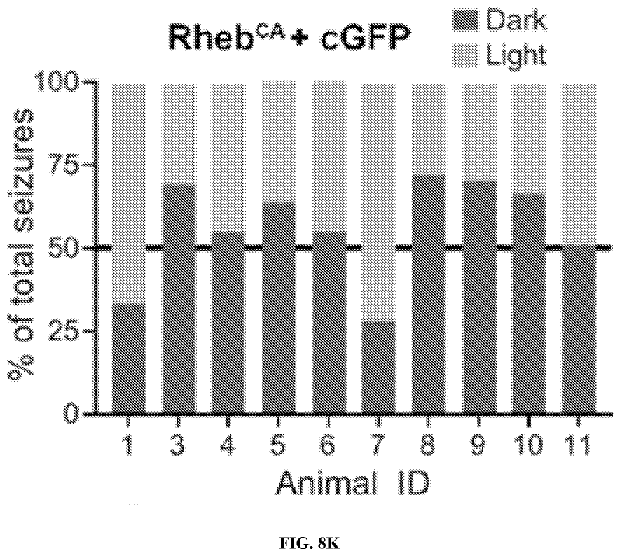

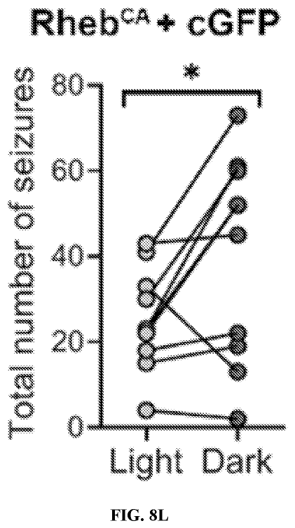

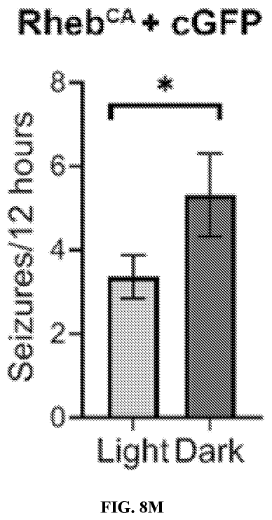

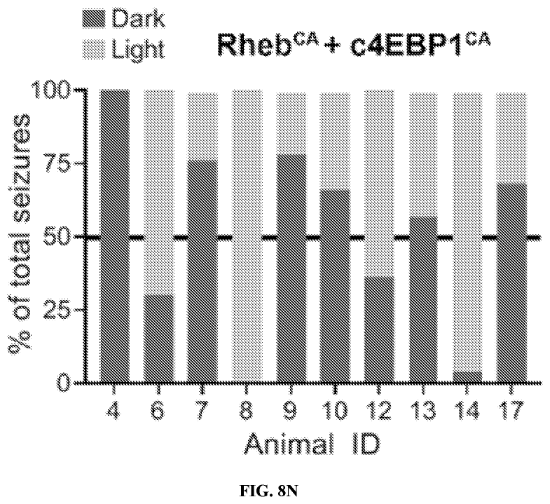

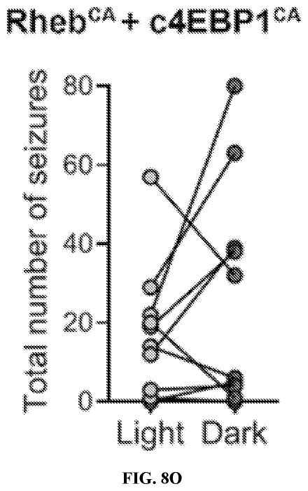

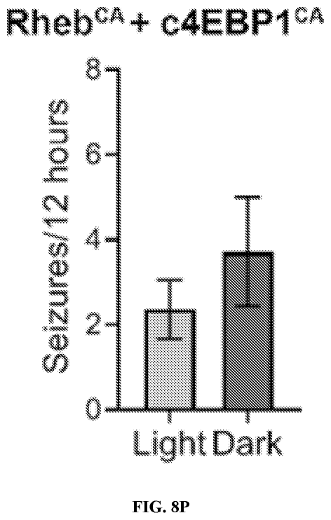

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, “4EBP1” refers to the protein having the sequence:

SEQ ID NO: 1
MSGGSSCSQTPSRAIPATRRVVLGDGVQLPPGDYSTTPG
GTLFSTTPGGTRIIYDRKFLMECRNSPVTKTPPRDLPTIPG
VTSPSSDEPPMEASQSHLRNSPEDKRAGGE ESQFEMDI

for the human homolog.

As used herein, “4EBP-activating agent” refers to a compound or compounds that increase or in combination work to increase 4EBP1 or 4EBP2 or 4EBP3 expression or activity or both.

As used herein, “EIF4E” refers to the protein having the sequence:

SEQ ID NO: 2
MATVEPETTPTPNPPTTEEEKTESNQEVANPEHYIKHPLQ
NRWALWFFKNDKSKTWQANLRLISKFDTVEDFWALYN
HIQLSSNLMPGCDYSLFKDGIEPMWEDEKNKRGGRWLI
TLNKQQRRSDLDRFWLETLLCLIGESFDDYSDDVCGAV
VNVRAKGDKIAIWTTECENREAVTHIGRVYKERLGLPP
KIVIGYQSHADTATKSGSTTKNRFVV

for the human homolog.

As used herein, “EIF4E-depleting agent” refers to a compound or compounds that reduce or eliminate or in combination work to reduce or eliminate EIF4E expression or activity or both. Agents targetting the EIF4G may in various embodiments prevent the formation of the EIF4F complex, thereby reducing or eliminating the activity of EIF4E. Accordingly, in various embodiments the EIF4E-depleting agent may target EIF4G.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of 20% or ±10%, more preferably +5%, even more preferably +1%, and still more preferably +0.1% from the specified value, as such variations are appropriate to perform the disclosed methods.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage. CRISPR/Cas9 technology is a biochemical method for genome editing that allows targeted modification by removing, adding or altering sections of the DNA sequence.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “epilepsy” refers to a condition in which a person has recurrent seizures. A seizure is defined as an abnormal, disorderly discharging of the brain's nerve cells (i.e. neurons), resulting in a temporary disturbance of motor, sensory, or mental function.

As used herein, the term “focal cortical dysplasia type II” or “FCD type II” means a disorder of brain development that leads to focal (or discrete) malformations of the cortex with specific cytoarchitectural alterations including (but not limited to) mislamination and neuron dysmorphogenesis. FCD type II can also refer to the malformation itself.

As used herein, “FCM neurons” refers to diseased cells. The term may refer to neurons or cells that are located inside the FCM or outside the FCM and express increased mTOR activity.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

As used herein, the term “tuberous sclerosis complex” or “TSC” means a genetic disorder resulting from mutations in the gene TSC1 or TSC2 and leads to a spectrum of peripheral and neurological alterations, including, focal malformations of the cortex that are called cortical tubers.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

A “target site” or “target sequence” refers to a genomic nucleic acid sequence that defines a portion of a nucleic acid to which a binding molecule may specifically bind under conditions sufficient for binding to occur.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, “treating a disease or disorder” means reducing the frequency or the severity with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Methods of Treating Disease

Without meaning to be limited by theory, the invention is based in part on the discovery that targeting cap-dependent translation is an effective method of treating epilepsy and/or preventing seizures. In various embodiments, the seizures or epilepsy are associated with mTOR hyperactivity.

In one aspect, the invention provides a method of preventing seizures in a subject in need thereof, the method comprising contacting a target cell of the subject with an effective amount of a 4EBP-activating agent. In various embodiments, the 4EBP-activating agent comprises a viral vector comprising a polynucleotide encoding a constitutively active form of 4EBP (4EBP^{CA}). 4EBP^{CA} is a mutated form of 4EBP (4EBP1, 4EBP2 or 4EBP3) that cannot be phosphorylated by mTOR and thus remains attached to the mRNA and blocks translation.

Examples of mutations include (but are not limited to) mutations at: Thr 37/46, Thr 70, Ser 65 and Phe 114 in human 4EBP1. In various embodiments, the 4EBP-activating agent comprises a viral vector comprising a mutated form of EBP comprising mutations at one or more of these positions. In various embodiments the mutated form of EBP is 4Ala 4EBP1, which has all its mTORC1-sensitive phosphorylation sites mutated to alanines, and constitutively binds to eIF4E thus repressing translation. Increases in the amount of 4EBP1 competent to interact with eIF4E by expression of exogenous 4EBP1^{4ALA} or 4EBP^{IF114A} preventing binding of eIF4G to eIF4E and thus bock cap-dependent translation.

In various embodiments, the viral vector further comprises a tissue specific promoter. In various embodiments, the viral vector is an adenoviral vector or a lentiviral vector. In various embodiments, the target cell is a neuron. In various embodiments, the neuron displays focal cortical malformations. In various embodiments, the 4EBP-activating agent is directly injected into the brain of the subject. In various embodiments, the 4EBP-activating agent is directly injected at a site of focal cortical malformation neurons.

In another aspect, the invention provides a method of preventing seizures in a subject in need thereof, the method comprising contacting a target cell of the subject with an effective amount of a EIF4E-depleting agent. In various embodiments, wherein the EIF4E-depleting agent comprises a viral vector comprising a EIF4E or EIF4G-inhibitory polynucleotide. In various embodiments, the EIF4E-inhibitory polynucleotide is an EIF4E or EIF4G antisense oligonucleotide, an EIF4E or EIF4G small hairpin RNA (shRNA), an EIF4E or EIF4G small-interfering RNA (siRNA) or a CRISPR system comprising a guide RNA targeting EIF4E or EIF4G. In various embodiments, the viral vector further comprises a tissue specific promoter. In various embodiments, the viral vector is an adenoviral vector or a lentiviral vector.

In various embodiments, the EIF4E-depleting agent is dominant negative EIF4E or EIF4G. Dominant negative, as the term is used herein, means that the polypeptide binds but does not perform its biological function. Accordingly, dominant negative variants interfere with the activity of their wild-type counterparts by excluding the wild-type molecule from its native binding site. In various embodiments, the dominant negative EIF4E or EIF4G is generated by CRISPR induced mutation or delivered by viral vector and overexpressed. Mutations that produce dominant negative phenotype are known in the art. In various embodiments the mutations that produce the dominant negative phenotype are: W43L, W46L, W113L, W130L, W166L, H200A or G111A.

In various embodiments, the target cell is a neuron. In various embodiments, the neuron displays focal cortical malformations. In various embodiments, the EIF4E-depleting agent is directly injected into the brain of the subject. In various embodiments, the EIF4E-depleting agent is directly injected at a site of focal cortical malformation neurons.

In various embodiments, the EIF4E-depleting agent is a EIF4E inhibitor selected from the group consisting of: Bn7GMP, 4Ei-1, 4EGI-1 and 4E1RCat.

### CRISPR/Cas

Genome editing using programmable nucleases enables precise editing at specific genomic loci, which can be used to remove deleterious mutations or insert protective mutations. To date, there are three major classes of nucleases—zinc finger nucleases (ZFNs), transcription activator-like effector nucleases (TALENs), and clustered, regularly interspaced, short palindromic repeat (CRISPR)-associated nucleases. Of these, CRISPR-associated nucleases have proven to be markedly superior to the others in terms of the ease and simplicity of use.

The CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The Cas9 protein, under direction from the gRNA, binds to its target DNA sequence and cuts both strands of the DNA at a specific locus. This double-stranded DNA break is repaired by either non-homologous end joining (NHEJ) or homology-directed repair (HDR). NHEJ frequently causes small insertions or deletions (indels) at the breakage site that can lead to a frameshift mutation of the protein encoded by the gene. HDR utilizes a repair template that is copied into the gene, thus engineering specific mutations. The CRISPR/CAS system can thereby be engineered to cleave virtually any DNA sequence by redesigning the gRNA.

One example of a CRISPR/Cas system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. The CRISPR/CAS system can also simultaneously target multiple genomic loci by co-expressing a single Cas9 protein with two or more gRNAs, making this system uniquely suited for multiple gene editing or synergistic activation of target genes.

In various embodiments, the nucleic acid capable of decreasing expression of the endogenous gene or a portion thereof is a CRISPR system. In some embodiments, the CRISPR system includes a Cas expression vector and a guide nucleic acid sequence specific for the endogenous gene. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

The guide nucleic acid sequence is specific for a gene and targets that gene for Cas endonuclease-induced double strand breaks. The sequence of the guide nucleic acid sequence may be within a locus of the gene. In various embodiments, the target gene is EIF4E or EIF4G. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

Hyperactivation of mTOR signaling due to mutations in mTOR regulatory genes is associated with malformations of cortical development and intractable epilepsy. mTOR regulates many key functions involved in cell growth but it is unclear which processes—contribute to epilepsy. Since current mTOR inhibitors such as rapamycin can have side effects, do not fully block all of mTOR's functions, and display limited efficacy, a more specific understanding of the downstream mechanisms by which mTOR contribute to epilepsy is crucial to inform and improve treatment options. mTOR complex 1 (mTORC1) regulates many cellular processes, with the best-studied function being cap-dependent translational control. mTORC1 activation promotes cap-dependent translation via inactivation of the translational suppressor 4EBP. In pathological conditions of mTORC1 hyperactivation, proper translation is disrupted. We previously showed that normalizing cap-dependent translation via expression of constitutive active 4EBP1 (4EBP1^{CA}) prevents mTORC1-induced cytoarchitectural abnormalities in mice, but the effects on seizures are unknown. Here, we evaluated whether decreasing cap-dependent translation suppresses seizures in mice with hyperactive mTOR signaling.

Our lab previously developed an in utero electroporation (IUE)-based mouse model in which constitutively active Rheb (Rheb^{CA}), the canonical activator of mTORC1, is expressed in developing cortical neurons, leading to mTORC1 hyperactivation, focal cortical malformations, and seizures. To evaluate the effects of reducing translation in this model, we performed TUE at embryonic day 15.5, targeting layer 2/3 pyramidal neurons. Half of the litter received a mixture of plasmids encoding Rheb^{CA}, conditional 4EBP1^{CA} (c4EBP^{CA}) and CreER, while the other half (control) received conditional GFP (cGFP) instead of c4EBP1^{CA}. Mice were treated with tamoxifen at 4 weeks of age to induce expression of the conditional plasmids. The resulting effects on seizures were monitored with continuous video-EEG recording for 7 days starting at 12 weeks of age. The expression and function of the 4EBP1^{CA} plasmid were validated by co-transfecting Rheb^{CA} and 4EBP1^{CA} plasmids in HEK cells and performing dual luciferase reporter assays and western blots to measure cap-dependent translation and protein levels of the mTOR pathway markers phospho-S6 (S240/244, mTORC1) and phospho-AKT (S473, mTORC2), respectively. We found that 4EBP1^{CA} expression decreased mTORC1-induced cap-dependent translation without altering phospho-S6 or phospho-AKT levels in HEK cells (n=3 replicates, P=0.0025 by Student's t-test). Importantly, reducing translation via 4EBP1^{CA} expression significantly reduced mTOR-induced seizure frequency by 60% in adult Rheb^{CA} mice compared to control (n=11-18 mice/group, P=0.0260 by Mann-Whitney test). Moreover, 8/18 (44.4%) Rheb^{CA}+4EBP1^{CA}mice did not display seizures compared to 1/11 (9.1%) mice in control group. (See FIGS. 10A-10P.)

Our findings indicate that targeting cap-dependent translation is sufficient to reduce seizures in mTOR-related epilepsy. These findings support altered cap-dependent translation as a crucial contributor to mTOR-induced epilepsy and targeting translation may be a more specific therapeutic strategy. Future studies aim to identify distinct molecules regulated by the mTORC1-4EBP1 pathway that contribute to the seizure mechanisms.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Enumerated Embodiments

The following enumerated embodiments are provided, the numbering of which is not to be construed as designating levels of importance.

Embodiment 1 provides a method of preventing seizures in a subject in need thereof, the method comprising contacting a target cell of the subject with an effective amount of a 4EBP-activating agent.

Embodiment 2 provides the method of embodiment 1, wherein the 4EBP-activating agent comprises a viral vector comprising a polynucleotide encoding a constitutively active form of 4EBP (4EBP^{CA}). Embodiment 3 provides the method of any of embodiments 1-2, wherein the 4EBP-activating agent is a viral vector comprising a CRISPR system configured to mutate 4EBP into a constitutively active form.

Embodiment 4 provides the method of any of embodiments 1-3, wherein the viral vector further comprises a tissue specific promoter.

Embodiment 5 provides the method of any of embodiments 1-4, wherein the viral vector is an adenoviral vector or a lentiviral vector.

Embodiment 6 provides the method of any of embodiments 1-5, wherein the target cell is a neuron.

Embodiment 7 provides the method of any of embodiments 1-6, wherein the subject displays focal cortical malformations.

Embodiment 8 provides the method of any of embodiments 1-7, wherein the 4EBP-activating agent is directly injected into the brain of the subject.

Embodiment 9 provides the method of any of embodiments 1-8, wherein the 4EBP-activating agent is directly injected at a site of focal cortical malformations.

Embodiment 10 provides a method of preventing seizures in a subject in need thereof, the method comprising contacting a target cell of the subject with an effective amount of a EIF4E-depleting agent.

Embodiment 11 provides the method of embodiment 10, wherein the EIF4E-depleting agent is dominant negative EIF4E.

Embodiment 12 provides the method of any of embodiments 10-11, wherein the EIF4E-depleting agent comprises a viral vector comprising a EIF4E-inhibitory polynucleotide.

Embodiment 13 provides the method of any of embodiments 10-12, wherein the EIF4E-inhibitory polynucleotide is an EIF4E or EIF4G antisense oligonucleotide, an EIF4E or EIF4G small hairpin RNA (shRNA), an EIF4E or EIF4G small-interfering RNA (siRNA) or a CRISPR system comprising a guide RNA targeting EIF4E or EIF4G.

Embodiment 14 provides the method of any of embodiments 10-13, wherein the viral vector further comprises a tissue specific promoter.

Embodiment 15 provides the method of any of embodiments 10-14, wherein the viral vector is an adenoviral vector or a lentiviral vector.

Embodiment 16 provides the method of any of embodiments 10-15, wherein the target cell is a neuron.

Embodiment 17 provides the method of any of embodiments 10-16, wherein the neuron displays focal cortical malformations.

Embodiment 18 provides the method of any of embodiments 10-17, wherein the EIF4E-depleting agent is directly injected into the brain of the subject.

Embodiment 19 provides the method of any of embodiments 10-18, wherein the EIF4E-depleting agent is directly injected at a site of focal cortical malformations.

Embodiment 20 provides the method of any of embodiments 10-19, wherein the EIF4E-depleting agent is a EIF4E inhibitor selected from the group consisting of. Bn7GMP, 4Ei-1, 4EGI-1 and 4E1RCat.

Embodiment 21 provides the method of any of embodiments 1-20, wherein the seizures are associated with mTOR hyperactivity.

What is claimed is:

1. A method of preventing seizures in a subject in need thereof, the method comprising contacting a target cell of the subject with an effective amount of a 4EBP-activating agent.

2. The method of claim 1, wherein the 4EBP-activating agent comprises a viral vector comprising a polynucleotide encoding a constitutively active form of 4EBP (4EBP^{CA}).

3. The method according to claim 1, wherein the 4EBP-activating agent is a viral vector comprising a CRISPR system configured to mutate 4EBP into a constitutively active form.

4. The method according to claim 2, wherein the viral vector further comprises a tissue specific promoter.

5. The method according to claim 1, wherein the viral vector is an adenoviral vector or a lentiviral vector.

6. The method according to claim 1, wherein the target cell is a neuron.

7. The method according to claim 6, wherein the subject displays focal cortical malformations.

8. The method according to claim 1, wherein the 4EBP-activating agent is directly injected into the brain of the subject.

9. The method of claim 8, wherein the 4EBP-activating agent is directly injected at a site of focal cortical malformations.

10. A method of preventing seizures in a subject in need thereof, the method comprising contacting a target cell of the subject with an effective amount of a EIF4E-depleting agent.

11. The method according to claim 10, wherein the EIF4E-depleting agent is dominant negative EIF4E.

12. The method of claim 10, wherein the EIF4E-depleting agent comprises a viral vector comprising a EIF4E-inhibitory polynucleotide.

13. The method according to claim 12, wherein the EIF4E-inhibitory polynucleotide is an EIF4E or EIF4G antisense oligonucleotide, an EIF4E or EIF4G small hairpin RNA (shRNA), an EIF4E or EIF4G small-interfering RNA (siRNA) or a CRISPR system comprising a guide RNA targeting EIF4E or EIF4G.

14. The method according to claim 13, wherein the viral vector further comprises a tissue specific promoter.

15. The method according to claim 13, wherein the viral vector is an adenoviral vector or a lentiviral vector.

16. The method according to claim 10, wherein the target cell is a neuron.

17. The method according to claim 16, wherein the neuron displays focal cortical malformations.

18. The method according to claim 10, wherein the EIF4E-depleting agent is directly injected into the brain of the subject.

19. The method of claim 18, wherein the EIF4E-depleting agent is directly injected at a site of focal cortical malformations.

20. The method of claim 10, wherein the EIF4E-depleting agent is a EIF4E inhibitor selected from the group consisting of: Bn7GMP, 4Ei-1, 4EGI-1 and 4E1RCat.

21. The method according to claim 1, wherein the seizures are associated with mTOR hyperactivity.
